(19) United States
(12) Patent Application Publication
(10) Pub. No.: US 2003/0074010 A1
(43) Pub. Date: Apr. 17, 2003

(54) Nanoscale explosive-implosive burst generators using nuclear-mechanical triggering of pretensioned liquids

(76) Inventor: Rusi P. Taleyarkhan, Knoxville, TN (US)

Correspondence Address: AKERMAN, SENTERFITT & EDISON, P.A., Suite 400, 222 Lakeview Avenue, P.O. Box 3188, West Palm Beach, FL, 33402-3188, US

(21) Appl. No.: 09/981,512

(22) Filed: Oct. 17, 2001

(51) Int. Cl. A61B 17/22

(52) U.S. Cl. 606/128

(57) ABSTRACT

A burst generator includes a structure for placing at least a portion of a liquid into a tension state, the tension state being below a cavitation threshold of the liquid. The tension state imparts stored mechanical energy into the liquid portion. A structure for cavitating provides energy to the liquid portion sufficient to bubble nucleate at least one bubble having a bubble radius greater than a critical bubble radius of the liquid, formation of the bubble releasing at least a portion of the energy which is stored in the tension state.

### STATEMENT REGARDING FEDERALLY SPONSORED RESEARCH OR DEVELOPMENT

The United States Government has rights in this invention pursuant to Contract No. DE-AC05-000R22725 between the United States Department of Energy and UT-Battelle, LLC.

### CROSS-REFERENCE TO RELATED APPLICATIONS

Not applicable.

### FIELD OF THE INVENTION

This invention relates generally to apparatus and methods for burst generation using liquids.

### BACKGROUND

It has been known for some time, although not widely recognized, that liquids, like solids, can be put under negative pressure. A condition of negative pressure is generally referred to as a state of tension, the tension state being a condition opposite to a state of compression. For example, a tree passes water upward against the force of gravity from its roots to its leaves. Since the hydrostatic pressure must decrease with height because of the force of gravity, the water pressure in trees reduces by 1 bar (approximately 15 psi) for approximately every 10 meters in height. The pressure of water at the top of a 100 m tall California redwood can be calculated as being approximately −9 bar (−132 psi). However, liquids such as water, cannot be tensioned without reaching a tension limit. Upon reaching a critical tension state, liquids fracture through the process of cavitation and release a portion of stored potential energy associated with the previous tension state upon the transition from liquid to the vapor phase.

Cavitation can be defined as the formation, growth, and collapse of vapor bubbles in a liquid. Cavitation can be forced to occur in a variety of ways, such as by sound waves, ultrasonic/acoustic waves, lasers, and by hydrodynamics.

Cavitation is a known phenomena which occurs when the pressure of a liquid is lowered to the point where a liquid starts to boil into a vapor, the local pressure being lower than the vapor pressure of the liquid. This process frequently occurs with marine propellers and pumps. In boating, cavitation occurs when a propellor is turned at a fast enough rate in water such that the water flowing over the propellor blade vaporizes.

Another example of cavitation relates to the cavitation of blood in humans.

Cavitation causes the familiar clicking sounds that are heard when knuckles are “cracked”. The tension of moving joints places blood under sufficient tension to cause cavitation bubbles to initiate beyond a certain tensile state. The bubbles collapse thereafter and shock waves generated by the collapse cause the familiar cracking sounds produced.

Cavitation has been used to break up kidney stones using lithotripters in a process referenced to as lithotriptcy. Lithotripters cavitate bodily fluids and send large magnitude (approximately 70 MPa) shock waves through bodily fluids that can be used to break up solid objects, such as kidney stones. However, 70 Mpa shock waves can also unintentionally damage surrounding tissue.

Some have used cavitation to systematically create and control certain processes. For example, controlled energy release from cavitation has been applied to provide relatively low level energy releases for the shear mixing of liquids and slurries and to aid in the synthesis of some materials.

### SUMMARY OF THE INVENTION

A burst generator includes a structure for placing at least a portion of a working liquid into a tension state. The tension state is below a cavitation threshold of the liquid and imparts stored mechanical potential energy into the liquid portion. A structure for cavitating the liquid portion provides sufficient energy to bubble nucleate at least one bubble having a bubble radius greater than a critical bubble radius of the liquid used. The formation of bubbles releases at least a portion of the energy stored in the tension state.

Tensioning the liquid can be provided by an acoustical source, an electrostrictive (piezoelectric) source, a magnetostrictive source, a centrifugal source or an acoustic wave source. Preferably, when an acoustical wave source is used, the acoustical source includes an acoustical focusing device, such as a parabolic-type reflector.

The structure for cavitating the liquid can be an acoustical source, or a source of fundamental particles, such as alpha emitters, neutron sources and fission fragment sources. When an acoustical source is used for tensioning, the same acoustical source (or one or more additional acoustical sources) can be used for cavitating the liquid. Alternatively, the structure for cavitating can be a laser source or a mechanical source. Energy stored in the tension state can be released rapidly, such as no more than 1.0 μsec following receipt of cavitation initiation energy from the structure for cavitating.

If a neutron source is used for cavitating the liquid, the neutron source is preferably an isotopic source having at least one shutter. The shutter can be opened to synchronize neutron impact with a location in the liquid when the liquid is at a predetermined liquid tension level. The working liquid can be chosen from a broad class of organic and inorganic compounds. For example, the liquid may be water, mercury, acetone, tetrachloroethylene, acetophenone (C₈H₈O) or various glycols, such as 1,2-propylene glycol or 1,3-propylene glycol. The working liquid can be a biological liquid, such as blood, synovial liquid, mucus and urine.

The structure for tensioning can include a controller for controlling the tension level in the liquid. A structure for generating an oscillatory pressure field can be provided in the liquid, where a compressive phase of the pressure field implodes at least one bubble formed.

The burst generator can include a structure for condensing working liquid vapor back into a liquid state following bubble formation. The burst generator can also include a cooling device for reducing a temperature of the liquid below ambient temperature. A controller can be provided for synchronizing delivery of at least one cavitation signal from the structure for cavitating at a predetermined location in the liquid, the predetermined location having a predetermined tension level.

In another embodiment of the invention, a burst generator includes a container for confining a working liquid, a structure for placing at least a portion of the liquid into a deep metastable state, and structure for cavitating at least a portion of the liquid sufficient to bubble nucleate at least one bubble having a bubble radius greater than a critical bubble radius of said liquid. The “deep metastable state” represents a set of conditions which approach, but do not exceed conditions required for homogeneous flash nucleation of the particular working liquid into a vapor state. The structure for placing at least a portion of the liquid into a deep metastable state can be an acoustical source and the structure for cavitating the metastable working liquid can be a neutron source.

An armament, such as a gun or a rifle, includes a substantially enclosed container having at least one projectile and a structure for placing a working liquid into a tension state, the tension state being below the cavitation threshold of the liquid. The working liquid can be water, mercury, acetone, tetrachloroethylene, acetophenone or glycols. A structure for cavitating at least a portion of the tensioned liquid sufficient to bubble nucleate at least one bubble having a bubble radius greater than a critical bubble radius of said liquid is provided. The structure for cavitating can be a source of fundamental particles, such as a neutron source. The formation of bubbles releasing at least a portion of the energy stored in the tension state, whereby an explosive burst results which propels the projectile out of container. The projectile can include an explosive.

The armament can include a structure to condense vapor back into a liquid state following bubble formation. A controller can be provided for controlling the thrust developed by the armament within the container, providing the ability to reach different distances without adjusting the firing angle of the armament. The thrust level can be controlled by controlling the level of the tension state and/or the energy imparted to the liquid by the structure for cavitating.

The armament can include a controller for synchronizing delivery of at least one initiation signal from the structure for cavitating with a desired tension level in the liquid. The armament can also include a cooling device for reducing the temperature of the liquid below ambient temperature.

A medical device includes a structure for placing a bodily liquid region contained within a body into a tension state, the tension state being below the cavitation threshold of the bodily liquid and imparting stored mechanical potential energy into the liquid. The bodily liquid can be blood, synovial liquid, mucus or urine. The structure for initiating cavitation produces at least one bubble while the bodily liquid region is in the tension state and then applies a compressive wave to collapse the bubble to cause implosion.

The structure for placing a liquid region contained within a body into a tension state and applying a compressive wave can be supplied by a single oscillatory pressure field source, such as an acoustic wave source. The acoustical wave source can include an acoustical wave focusing device, such as a parabolic-type reflector.

The structure for initiating cavitation can be an acoustical source, or a source of fundamental particles, such as an alpha emitter, neutron source and fission fragment source. The neutron source can be an isotopic source having at least one shutter. The shutter can be opened to synchronize neutron impact with a location in the bodily liquid having a predetermined liquid tension level. Alternatively, the structure for cavitating can be a laser source or a mechanical source.

The structure for placing a bodily liquid into a tension state can include a structure for controlling a level of the tension state. Bodily liquids can be placed in an oscillating pressure state distribution by using wave superposition, the waves provided by a plurality of oscillatory pressure sources.

A pulse generator includes a container for containing a working liquid and placing the liquid into a tension state, the tension state being below the cavitation threshold of the liquid. The working liquid can be water, mercury, acetone, tetrachloroethylene, acetophenone or glycols. A structure for cavitating at least a portion of the tensioned liquid can provide sufficient energy to bubble nucleate at least one bubble having a bubble radius greater than a critical bubble radius of the liquid, formation of the bubble releasing at least a portion of the energy stored in the tension state.

The structure for placing a liquid under tension can be an acoustic wave source. The acoustical source can also function as the structure for cavitating the working liquid. The structure for cavitating the working liquid can be a source of fundamental particles, such as a neutron source. The neutron source can be an isotopic source having at least one shutter. That shutter can be adapted to open at a time to synchronize neutron impact with a location in the liquid having a predetermined liquid tension level.

The structure for placing a liquid into a tension state can include a structure for controlling the level of the tension state. The pulse generator can include a structure for converting generated bursts into electromagnetic signals, such as optical or electrical signals. Bursts from the pulse generator can be directed to propel a liquid through an orifice, such as in a MEMS device, the orifice being no larger than micron scale.

A method for producing energetic bursts includes placing a working liquid into a tension state, the tension state being below the cavitation threshold of the liquid. The working liquid can be water, mercury, acetone, tetrachloroethylene, acetophenone or glycols. The working liquid can be cooled to a temperature below ambient temperature.

At least a portion of the tensioned liquid is cavitated with sufficient incident energy to bubble nucleate at least one bubble having a bubble radius greater than a critical bubble radius of the liquid. Bubbles produced release at least a portion of the energy stored in the tension state. Bursts can be explosive or implosive. Implosive bursts can be generated by applying compressive pressure field to bubbles formed in the working liquid.

A centrifugal source and an acoustic wave source can be used for tensioning. When an accoustical source is used for tensioning, the method can include the step of focusing the acoustical waves. A parabolic-type reflector can be used for focusing.

A source of fundamental particles, such as a neutron source can be used for the cavitating step. Neutron impact can be synchronized with a location in the working fluid having a predetermined liquid tension level.

### BRIEF DESCRIPTION OF THE DRAWINGS

A fuller understanding of the present invention and the features and benefits thereof will be accomplished upon review of the following detailed description together with the accompanying drawings, in which:

FIG. 1 is a schematic illustration of an embodiment of the invention.

FIG. 2 is a plot of the variation of normalized homogeneous nucleation temperature with pressure.

FIG. 3 is a schematic illustration of a centrifugal apparatus for tensioning a liquid according to an embodiment of the invention.

FIG. 4 is a plot of pretension pressure versus rotation speed and arm length for a centrifugal apparatus.

FIG. 5 is a plot of pressure versus time for liquids attaining variably-explosive or implosive forces.

FIG. 6 is a schematic illustration of an acoustic lens-neutron generator source system, according to an embodiment of the invention.

### DETAILED DESCRIPTION OF THE PREFERRED EMBODIMENTS

The invention includes methods and apparatus for placing a liquid into a metastable state, such as a tension state, and applying energy to cavitate the liquid to quickly and controllably release a portion of the energy stored in the metastable state. For example, if liquid tensioning is used, a tension state is first reached which is below a cavitation threshold for the liquid. A tension state imparts stored mechanical energy into a portion of a liquid, or can impart mechanical energy into the entire liquid volume. Liquid tensile states are one example of a metastable state (as defined below).

In the case of tensioning, after the metastable state is reached, at least one initiation signal from a source of cavitation energy is directed to the metastable liquid portion. The energy signal provides sufficient energy to cavitate the liquid through bubble nucleation of at least one bubble having a bubble radius greater than a critical bubble radius for the specific liquid used. Formation of bubbles of at least the critical size results in bubble growth and can lead to the release of at least a portion of the energy stored by the liquid in the metastable state.

A classical system is considered to be in a metastable state if it is in a state above its minimum-energy state, but requires an energy input before it can reach a lower-energy state. For example, a superheated liquid, such as pure water at 110° C. at one atmosphere pressure, is in a metastable state since its lower energy state is a vapor state. A metastable system can act as a pseudo-stable system, provided that energy inputs, such as from thermal or mechanical sources supplied to the system remain below some activation threshold. Systems with strong metastability are commonly described as being stable systems. An associated, broader definition of metastable embraces all systems that have a long lifetime (by some standard in an energy state above its minimum-energy state.

An objective standard for a metastable state can be defined to be a fluid state where homogeneous self-nucleation of bubbles due to statistical fluctuations can grow uncontrollably at the limit of homogeneous nucleation. The time involved for such an effect is on the order of nanoseconds. Therefore, metastable states not at the limit of self-nucleation from statistical fluctuations generally involve time frames that are orders of magnitude longer than the nanosecond range. Upon application of suitable cavitation initiation energy to the metastable liquid, such as from neutrons, alpha particles or laser beam heating, the growth to critical bubble radii can be in the nanosecond range.

As used herein, the phrase “deep metastable state” represents a set of conditions which approach, but do not exceed conditions required for homogeneous flash nucleation of the particular working liquid into a vapor state. The attainment of a deep metastable state imparts significant potential energy into a suitable working liquid which can be released upon application of an appropriate energetic signal from a cavitation initiation source. The deeper the metastable state, the less energy is required from the cavitation initiation source to bubble nucleate at least one bubble having a radius greater than the critical radius for the working liquid.

As described in detailed later, energy can be released upon explosion or implosion of cavitation bubbles formed from cavitating the metastable liquid to reach the lower energy state. Explosion of bubbles generated can produce propulsive, outward forces. If the explosion occurs over a very short period of time such as over a period of nanoseconds, bubble explosion can give rise to high-explosive type effects with very rapid expulsion of material surrounding the bubbles such that shock waves may form and the material expelled can be sheared apart and dispersed.

The propulsive energy that is produced by the explosion can be harnessed to propel projectiles. If controlled and appropriate parameters applied, the cavitation process can be used to designate a desired pressure profile behind a projectile to independently produce a desired projectile range.

The invention can also be used to generate implosive collapse of cavitation bubbles formed, such as through application of a compressive pressure field to cavitation bubbles. Implosive bubble collapse generates localized shock waves and can generate extremely high temperatures and pressures. Localized regions of high pressure and temperature can be used in medical applications to destroy cells such as cancer cells and to erode or otherwise pulverize substantially solid materials, such as kidney stones.

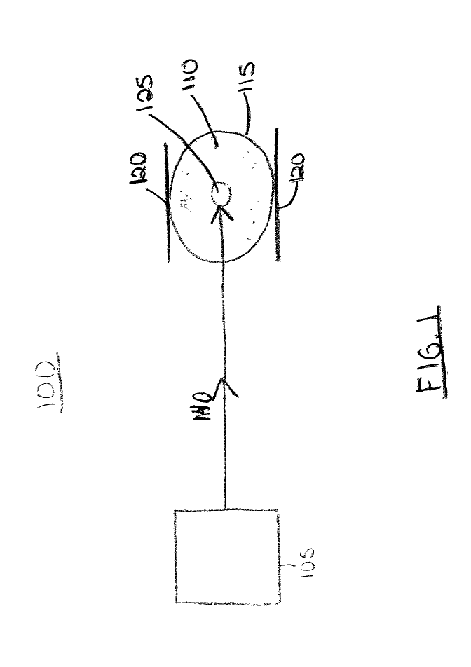

The cavitation of metastable liquids can produce explosive or implosive vaporization and release of stored energy. A burst generation system 100 is shown in FIG. 1. A working liquid 110 is provided in a substantially enclosed volume 115, the working liquid having a suitably high cavitation threshold for the intended application. A structure 120 for placing the working liquid in a metastable state, such as a tension and/or a superheated state, is provided to produce metastable region 125 within working liquid 110. Laser pumping may be used to achieve a selected degree of superheat in the working liquid. The metastable state imparts stored potential energy, such as mechanical energy in the case of tensioning, into metastable liquid region 125.

As noted above, metastable states in liquids can be provided using several different methods, some of which may be combinable. The processes of pretensioning and superheating may be used, either separately, or together. The combination of these methods can be used to maximize energy available from the system for explosive or implosive release.

Cavitation initiation source 105 emits energetic particle or wave energy 140, the energy 140 directed to strike metastable region 125. Cavitation initiation source 105 directs cavitation source energy to the metastable liquid region 125 sufficient to bubble nucleate at least one bubble having a bubble radius greater than a critical bubble radius of the liquid. The critical bubble radius (r_{crit}) is a function of the pressure within the bubble (P_{bubble}), the ambient pressure (P_{ambient}) and the surface tension of liquid (a) and is given by the following equation:

r_{crit}=2σ/(P_{bubble}−P_{ambient})

The formation of at least one bubble having a radius of at least the critical radius (r_{crit}) given above permits the bubble to grow and leads to release of at least a portion of the energy stored by the metastable working liquid. The amount of resulting energy produced is controlled by the stored potential energy in the working liquid (e.g. pre-tension level) combined with the energy supplied by the initiation source 105.

The release rate of the stored energy in the metastable state can be controlled in both time and space based on the intended application by creation of an appropriate metastable state and selection of appropriate initiation source parameters. Optionally, in certain desired low speed processes (e.g. millisecond), the system can include propagation control devices such as scattering centers placed within the working liquid volume to reduce the resulting wave speed. Scattering centers scatter acoustic pressure waves which can result in a reduction of the wave propagation speed.

Preferred scattering materials have low impedance relative to the bulk working liquid. Accordingly, the product of density times sound velocity of the selected scattering center should be significantly lower, preferably by a factor of ten (10) or more, compared to that of the working liquid. Scattering centers may be a plurality of pre-positioned small particles or vacuum like cavities, such as bubbles. Particles can be provided in shapes such as spherical. The scattering centers can be glass or steel particles, and can have hollow centers. Only a small fraction, such as 1 to 5% of the working liquid volume, is generally required for significant a reduction in wave speed. For example, for an air-water system it is known that the sound velocity through water can be reduced by a factor of 10 or more when 1 to 2% of the volume comprises air bubbles.

The metastable state can be controlled with parameters such as temperature and pretensioning level, and the distribution of these parameters within the liquid volume. Initiation source parameters can be selected from parameters including timing of the application of initiation energy, initiation energy type, intensity and spatial coverage.

The initiation source 105 can be controlled to affect a small portion of a liquid 110 or the entire liquid 110. If neutrons are used, the beam of neutrons could be collimated to a desired size to affect a given size volume of liquid depending on how large or small a region is desired to be cavitated. For example, a cavitation process could be controlled with the number of nucleators emitted by the cavitation initiation source (e.g. neutrons) in conjunction with how far the system is from the homogeneous nucleation temperature.

In the case the system is in a deep metastable state being close to the homogeneous nucleation temperature, an energetic stimulus applied locally anywhere in the system would generally cause the entire liquid volume to participate in cavitation. The speed of this locally initiated process resulting in bulk participation generally depends on the sonic velocity of propagation of cavitation bubbles. Thus, to obtain relatively instantaneous (nanosecond) timing of the entire cavitation process, the system would generally be brought close to the homogeneous nucleation temperature and the entire volume would be subjected to initiation source energy, such as a beam of neutrons.

A longer duration (milliseconds) process does not generally require that the liquid be brought close to the homogeneous nucleation temperature as in the faster nanosecond process described above. The millisecond process could be started by initiating nucleation at one end of the liquid volume and initiating other portions of the liquid volume at a later time. The liquid can also be provided scattering centers therein or the liquid volume could have serpentine pathways if it is desired to slow the rate of cavitation propagation, as in a millisecond process.

To propel a projectile, the explosive vaporization process generally is desired to proceed in a controlled manner over a relatively long time period, the time period generally measured in milliseconds. The expanding bubbles can perform mechanical work, such as to propel a projectile such as a bullet in a gun. To create an explosive shock wave, the cavitation parameters should be chosen to complete the vaporization process within microseconds or less, causing effects similar to those from conventional high-explosives.

Applications which require localized shocks and intense pressure-temperature buildup can be obtained by controlling the expanding bubbles to attain ultra-fast collapse, such as over a period measured in nanoseconds. Such collapses can be intense enough to result in the emission of visible flashes of light in a process known as sonoluminescence. This process, if conducted with suitable liquids, such as liquids capable of sustaining high levels of tensioning prior to fracture, can produce a very large energy density, having energy density factors of up to 10⁶ greater than that possible from conventional high-energy density materials (HEDM), such as CHNO compounds.

A wide variety of working liquids can be used with the invention. Using cooling techniques such as refrigeration, substances which are gaseous under standard temperature and pressure (STP) can be forced into a liquid state and become potentially useful with the invention. When tensioning is used to create the metastable state, liquids are generally selected which have high pretension states prior to fracturing. The higher the pretension state available prior to fracture, the larger the available energy for release upon vaporization. Experiments for determination of thresholds for the fracture of pretensioned fluids under various states have been conducted at Oak Ridge National Laboratory (ORNL) [1] and elsewhere [2,3].

Generally, the presence of impurities and dissolved gases causes liquids to cavitate with only modest applied tensions. This would make these liquids generally unsuitable for use with the invention. For example, cavitation of ordinary tap water has been demonstrated with only a few psi of tension, due to impurities in the water. [4]. However, the maximum possible extent of pretensioning a liquid prior to onset of cavitation for a substantially purified low density liquid such as water, and a high density liquid such as mercury can be quite large [5, 6]. Purified or distilled water may reach approximately −1,400 bar (−20,000 psi) and mercury −17,000 bar (−250,000 psi).

It is useful to compare the above attainable tensile states with pressures reached in conventional firearms, such as guns. In shotguns, the transient pressure levels are typically 700 bars (10,000 psi) and about 2100 bars (30,000 psi) in high-velocity rifle cartridges. Thus, pretensioned liquid states have the potential to deliver comparable or larger values of propulsive force compared to conventional propulsive materials. Suitable pretensioned liquids can also be selected which are largely unreactive, such as water, and avoid reactive complex nitrogen based HEDMs which are conventionally used.

Water, even only at 1 bar and slightly above saturation temperature possesses close to 2.5 MJ/kg of stored energy in terms of the energy released upon flash vaporization from liquid to vapor. With larger tension levels in the 100+ bar and higher range possible using purified or distilled water, it is estimated that the stored energy levels (and mechanical work capabilities) can be raised approximately 20 MJ/kg, or more. The precise value of mechanical work capability which can be generated by the system depends on factors such as system design and the method and parameters used to initiate cavitation. This level can be compared with energy levels of commonly used CHNO-type mixtures like TNT and HMX which are in the 4-6 MJ/kg range.

Overall, if properly initiated into cavitation and controlled, pre-tensioned liquids with controlled initiation of cavitation for nanosecond burst vaporization possess the potential for producing comparable or significantly higher levels of explosive energy per unit mass of material compared to conventional explosive materials. Moreover, some useful working liquids, such as water, may be inherently safe materials. Such materials can be used to limit or even avoid associated issues of toxic byproducts and/or use of complex specialized safety-related handling methods.

Energy available for burst generation can also be supplied or enhanced by superheating the working liquid. Pretensioning and superheating energy can be combined. In this case, the stored metastable energy can be the sum of stored energy from pretensioning plus energy stored in the superheated state.

Nuclear energetics, such as initiation of nuclear fusion reactions, can result from the cavitation process and increase the above energy level values by a factor of up to 10⁶. For example, implosive dynamics producible by the invention could be robust enough to lead to nuclear fusion. If so, deuterium-deuterium (D-D) or deuterium-tritium (D-T) nuclear reactions can take place. The energy density of release from D-D or D-T reactions is close to 10⁶ times greater than that available from conventional chemical explosives.

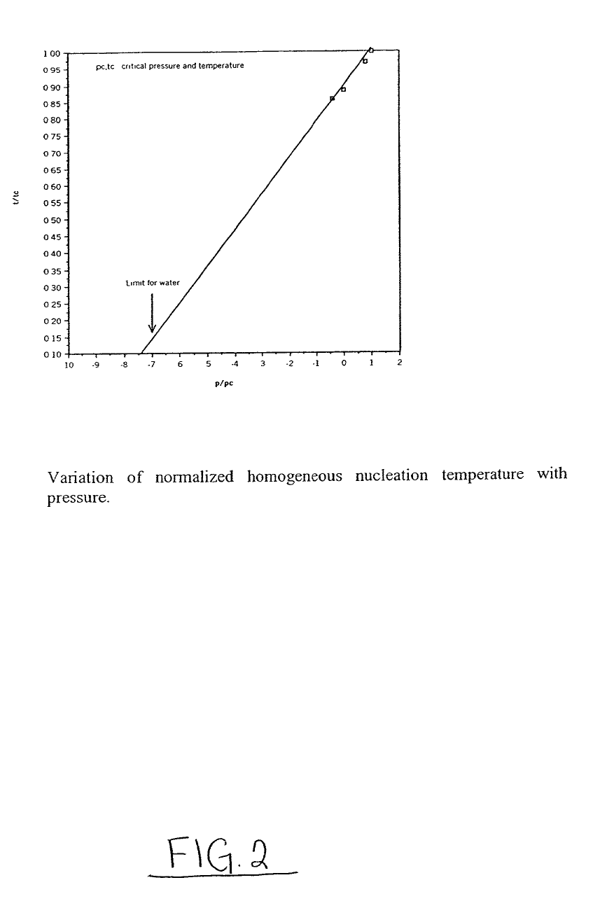

FIG. 2 shows the variation of the normalized homogeneous or spontaneous nucleation temperature with normalized pressure of the liquid field for water.

Normalization is relative to the critical point. The database involves extrapolation below the normalized pressure value of approximately −1. The extrapolation is based on a linear profile on adjacent portions of the plot. The greater the degree of pretension, the lower the homogeneous limit and the easier it becomes to destabilize the entire system to explode. As shown in FIG. 2, at a normalized negative pressure of −7 (i.e., −1400 bar) for water, the normalized temperature is only 0.14. In the absence of further information, the information shown in FIG. 2 could be used in a scaled manner for other fluids also since the underlying physics forming the basis of the data compiled in FIG. 2 should apply to other liquids.

Pretensioning of liquids to significant tension states has been demonstrated with tetrachloroethylene (C₂Cl₄) [7], and acetone (C₃H₆O) [8]. The same could be done for liquids ranging from pure relatively small atoms and molecules such as water, ethanol and mercury, to complex liquids such as the biological fluids blood, synovial liquid, mucus and urine. Intense tensile liquid states can be derived from a variety of liquids such as several inorganic and organic liquids. Preliminary choices include water, blood, urine, mercury, acetone, tetrachloroethylene, acetophenone and glycols, such as 1,2-propylene glycol or 1,3-propylene glycol. The working liquid can be a biological liquid, such as blood, synovial liquid, mucus and urine.

Liquids to be used could either be organic, inorganic or complex and could either be pure or suitably doped to form mixtures of liquids or pure liquids.

Nucleating agents can be used to enhance the bubble nucleation rate. For example, uranyl nitrate salts or a dissolved alpha emitter can be used for this purpose. Nucleating agents such as uranyl nitrate can function as emitters of fundamental particles, such as neutrons and alpha particles. Emissions from nucleating agents work in essentially the same manner as neutrons or optical beams from external sources, such as accelerator and isotope based neutron sources and lasers, respectively. These agents can be used to provide energy needed on a nano or macro scale to permit growth of cavities to reach the critical level.

Liquid and system preparation can be an important consideration in conjunction with use of pretensioning and cavitation initiation apparatus. The key parameters to be controlled are gas content, microscopic impurities, cleanliness of the structural components and system temperature. For attaining large metastable states, the liquids should be degassed, using methods such as ultrasonic agitation under vacuum, or via boiling.

Liquids are preferably purified, using filters such as micropore filters, to remove motes and inclusions. The internal structural surface conditions are preferably cleaned via acetone or other dissolving agents as needed. As the temperature is raised, the maximum attainable pretension level becomes lower.

Therefore, the liquid temperature could also be important to keep as low as practical via use of cooling air drafts or via other cooling techniques.

Depending on the intensity of metastable state (e.g. pretension) created, the bubbles that are nucleated can be of sufficient size to grow. The geometrical volume of influence, degree of growth and collapse of bubbles can be controlled using techniques identified in subsequent sections of this disclosure.

A variety of structures for placing the working liquid in a metastable state can be used with the invention. In the case of a tension state, the structure 120 for placing the working liquid in a metastable state produces a tension state in the working liquid which is below the cavitation threshold of the working liquid. If tensioning is desired, structure 120 can be a pressure wave source, an electrostrictive (piezoelectric) device or a centrifugal device. Pressure wave source can include acoustical sources, such as ultrasonic sources, mechanical sources, laser, or microwave sources. A pressure wave source preferably includes a wave focusing device, such as acoustical wave focusing device. For example, the focusing device can be a parabolic-type reflector.

In the case of tensioning, the tensioned volume capable of bulk explosive cavitation after application of appropriate energy from cavitation initiation source 105 will generally depend on the amplitudes of the pressures imparted to the working liquid by the structure for placing the working liquid in a metastable state 120. The larger the tension state amplitude attained, the greater the size of the sensitive region produced.

Acoustic source drivers, such as magnetostrictive or piezoelectric drivers, can generate an oscillatory pressure field in the working liquid, creating a tension state in discrete regions of the working liquid. Acoustically excited burst generation systems can possess the ability to store very large amounts of energy that can be released on demand by application of appropriate cavitation initiation energy.

The timing of application of cavitation initiation source energy, such as a neutron source, can be coordinated with the phase angle of the resulting pressure waves in working liquids. For example, the shutter of a neutron cavitation initiation source can be timed to open only at times when the working liquid is tensioned to the desired level.

The resulting pressure profile from an acoustical source will vary with the chamber shape and the number of source drivers used. However, using wave superpositioning of a plurality of waves, a variety of oscillating pressure profiles can be obtained. The pressure field obtained can also be a standing wave. For a simple cylindrical chamber geometry with only one driver the resulting wave shape is generally sinusoidal in the axial direction and a Bessel function in the radial direction.

The use of acoustics provides the capability to vary the frequency of tensile state attainment over a very wide range, such as several Hz up to tens of kHz. Low frequency operation can permit larger vapor cavity growths and collapse at reduced levels of pressure fluctuation. This occurs because the vapor cavities will provide additional time for bubble growth until arrested by the positive part of the pressure wave. On the other hand, if greater control of time is necessary, a combination of a higher pressure amplitude with higher frequencies may be employed. The inventor has demonstrated close to 1000 psi (70 bar) type oscillating pressures in a 1 L test chamber and has also demonstrated operation with multi-frequency excitation [9] wherein the transient pressure field can be composed of more than one pressure component.

Multiple pressure component permits superposition of the respective pressure fields. Superposition can provide resulting pressure fields which cannot be obtained from a single pressure component.

Other methods for introduction of metastable states in tension include Berthelot's method in which a working liquid is treated with a thermal cycle. In Bertholet's method a fluid in a given chamber is heated and permitted to expand past a restriction, such as a valve. Thereafter, the valve is closed and the fluid is allowed to cool down. Since the fluid is taking up the same volume with a reduced level of mass, the fluid undergoes tension. Another method for introducing a metastable tension state uses an explosive charge to decompress a compressed charge of liquid. As the piston drives off a rarefaction wave is transmitted through the mixture placing the fluid in tension to the extent of the degree of decompression.

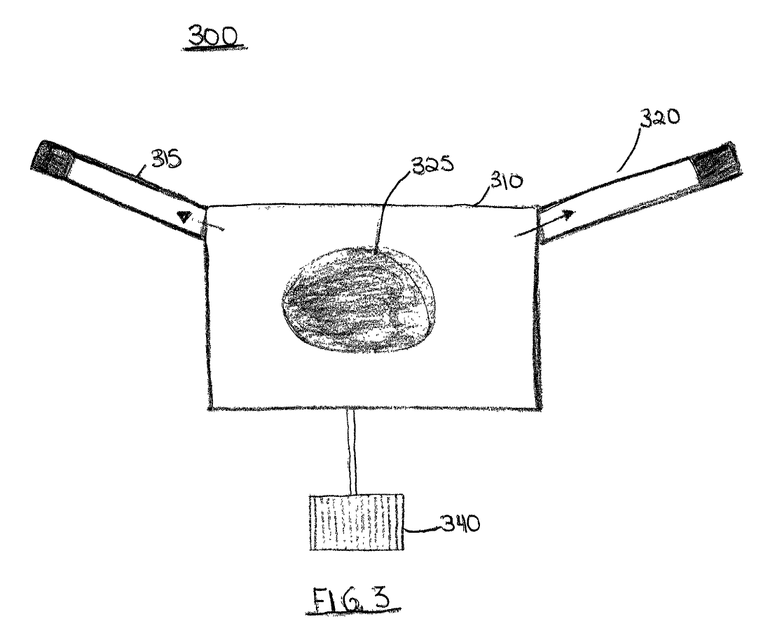

A centrifugal source can be used to produce significant levels of tension through the entire volume of a working liquid. FIG. 3 shows a centrifugal tensioning source 300 which can provide 360 degree projectile transport coverage with the added potential benefit of centrifugal forces at the projectile location. The projectile at the end of the arm can, upon release of the restraining boundary, be emitted outward from the arm due to centrifugal forces at a velocity equal to frequency (radians/per sec) of rotation times the radius from the central axis. The exploding vapor from the center can boost this centrifugal velocity as it receives stored up metastable energy released responsive to application of appropriate energy from a suitable cavitation initiation source.

Source 300 utilize a volumetric cavity 310 containing the working liquid from which energetic bursts are attained. Cavity 310 has at least one, but preferably having two or more arms, such as 315 and 320 shown stretching diametrically out of the central reservoir 325 to achieve balance.

Either a single or a mixture of liquids can be placed in reservoir 325 apparatus such that the liquid meniscus is just above the bends in the arms (not shown). System 300 when spun about its central axis develops uniform negative liquid pressures in the central reservoir 325. The pressure in the rotating arms varies from being at ambient pressure at the far side of the arm to the most negative at the interface with the central bulb. The pressure variation is described by the equation below:

ρ_{neg}=19.73×(fluid density)×(arm length)²×(angular rotation speed in HZ)²−ambient pressure

The spinning source could be driven by a conventional electric motor, such as motor 340. Alternatively, the system could be magnetically levitated and spun using conventional electromagnetic force fields.

The centrifugal source 300 shown in FIG. 3 displays inherent stability due to the liquid in the opposite arms 315 and 320 counterbalancing small system perturbations which can develop. Centrifugal source 300 can also develop high tensile pressure levels quite rapidly assuming use of a motor 340 having sufficient power to quickly reach the desired rotation speed, such as within several seconds. Centrifugal source working liquid can be filled on an as-needed basis from a charging reservoir (not shown). However, suitable structure (not shown) can be added to system 300 to return the working liquid back after deployment to the central reservoir 325 by condensing the working liquid following deployment. Deployment may include projectile escape from a burst system such as 100, the system 100 employing centrifugal tensioning source 300.

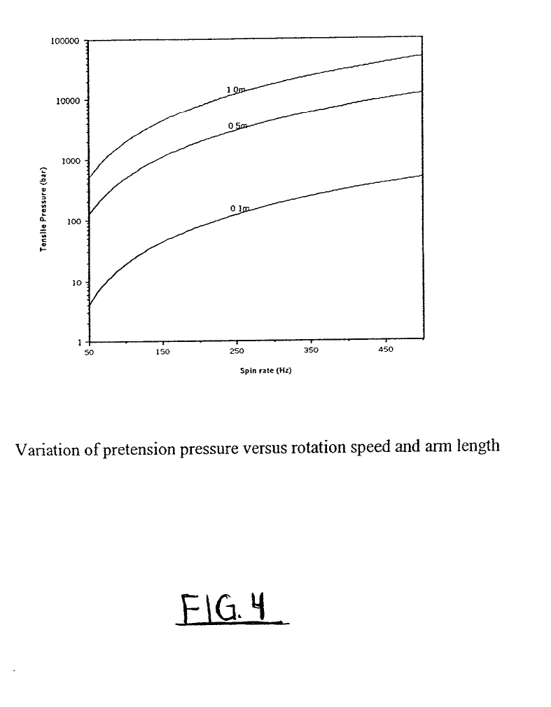

Various levels of pretension in water using centrifugal source 300 for a range of length and rotation speeds are shown in FIG. 4. Significant metastable levels can be obtained with a reasonable choice of system geometry and operating parameters. For example, approximately 1000 bar of pretension pressure is obtained from apparatus 300 having an arm length of 0.5 m at a spin rate of 150 Hz.

Source 300 can permit system 100 to provide a selectable phase angle and resulting direction of the resulting energetic burst following application of appropriate cavitation initiation energy at the proper time. Selection of a phase angle refers to the angle of rotation of the spinner (0 to 360 degrees) at which point cavitation of the metastable fluid in the central fluid region can be initiated. For example, selection of a phase angle enables launching a projectile at a predetermined angle in a gun system as the centrifugal source 300 rotates in a circular fashion.

The potential energy stored in a metastable liquid, such as a pre-tensioned liquid, can be released with application of appropriate cavitation initiation energy. The initiation energy source 105 supplies energy to destabilize the metastable liquid molecules to cause bubble nucleation of at least one bubble having a bubble radius greater than a critical bubble radius of the liquid. Formation of the above bubble releases at least a portion of the energy stored in the metastable state (e.g. tension state) resulting in explosive or implosive burst generation.

The dynamics of forming bubbles is dependent on the metastable state of the liquid which is achieved prior to cavitating. The higher the metastable energy level imparted to the working liquid, the lower the initiation energy required for bubble nucleation. In addition, the closer the metastable state is to that required for homogeneous nucleation, the larger will be inherent statistically-induced fluctuations in the liquid volume, again resulting in generally lower cavitation initiation energy requirements.

An appropriate initiation source 105 for applying cavitation source energy to the working liquid permits the realization of controlled process kinetics through the ability to initiate cavitation of metastable liquids on-demand and in a controlled manner. Metastable liquids can be fractured locally, such as on a nano-scale, or volumetrically within nanoseconds to microseconds depending on the initiation source and initiation source parameters used.

If nano-scale accuracy is desired, a variety of simple portable platforms for various length and time scales can be used. For example, ionizing particle and/or mechanical perturbation-based techniques can be used to initiation nucleation to produce nanosecond cavitation processes. Some ionizing particle sources, such as neutron sources, can be safe and lightweight. Such sources permit the configuration of portable burst systems 100 which can be easily carried by an individual.

Ionizing particle techniques utilize fundamental particles, such as neutrons, alpha particles or fission fragments. These particles have been demonstrated to be able to interact with individual nuclei of the target liquid atoms to permit nanosecond timed initiation of explosive vaporization. This invention can also utilize a variety of nucleating agents, such as dissolved alpha emitters, dissolved fissioning nuclei and the use of externally generated neutrons from small hand-held isotopic sources (such as californium or Pu—Be) or using pulsed neutron sources that are based on D-D and D-T reactions and produce 4 Mev and 14 Mev neutrons, respectively. Such sources of nucleating agents are readily available for safe use (with appropriate shielding).

When using pulsed neutron sources, neutrons are emitted only when the system is activated. Therefore, no additional shielding is needed. For isotopic neutron sources, the source could be contained in a container with a fast-opening shutter that is opened on demand to allow a burst of neutrons to emanate and enter the liquid region for flash vaporization to take place. In the case of tensioning, the timing of the shutter opening is preferably coordinated using an appropriate control system with the phase angle of rotation if a centrifugal apparatus is used, or the working liquid is tensioned to a desired tension level in the case of a resonant tensioning system.

The resulting bursts can take place either locally when a selected portion of the liquid is fractured such that the balance of the pre-tensioned liquid is propelled as projectiles, or when substantially the entire liquid volume undergoes homogeneous nucleation into the vapor phase and results in explosive phase change-induced power bursts. Explosive phase change-induced power bursts are turbo-charged by the intense pre-tension related pressure work component. It is also possible to control the timing of occurrence and the intensity level of the bursts by controlling the flux rate of the initiation energy applied.

If nanoscale localized initiation is desired, the nanoscale initiation of pretensioned liquids can be attained via targeting and subjecting individual nuclei of liquid molecules to knock-on collisions with fundamental particles, such as neutrons and alpha recoils. The collision of a high-energy (e.g., 14 Mev) neutrons with an individual nucleus results in recoil-induced deposition of thermal energy in a tiny (sub-nanometer) region of the targeted liquid. If this energy level if sufficient to cause a vapor nucleus to form with a radius larger than the critical radius of the liquid, the vapor cavity can grow. Critical radii are generally in the nanometer range and are formed within nanoseconds. For example, for an organic liquid such as C₃H₈, the critical radius is calculated to be about 0.9 nanometers with nucleation from alpha recoils using dissolved Po or energetic neutrons particles.

Laser beams may also be used as a cavitation initiation source 105, but extra hardware such as a laser source and mirrors would generally be needed and nanoscale control may be lost because of the available laser spot size. Microwave energy input could also be utilized along with mechanical shocks, the negative edge of the shock causing initiation by placing the liquid already in tension beyond the threshold.

The use of laser beams can negate the molecular scale triggering on a nanospace scale. However, larger beam sizes can be attained with devices such as a beam expander or mechanical perturbations for nucleating macro-scale regions which can grow on a nanosecond time scale to critical bubble sizes. Such a system could provide nanosecond control.

It is possible for a single source to provide both liquid tensioning and cavitation initiation energy for a burst generation system. For example, an acoustical source can be used to place the working liquid under tension. The same source can also be used to provide energy necessary to initiate cavitation of the tensioned working liquid. Alternatively, a first acoustical source can provide tensioning while a second acoustical source is used to provide energy to initiate cavitation.

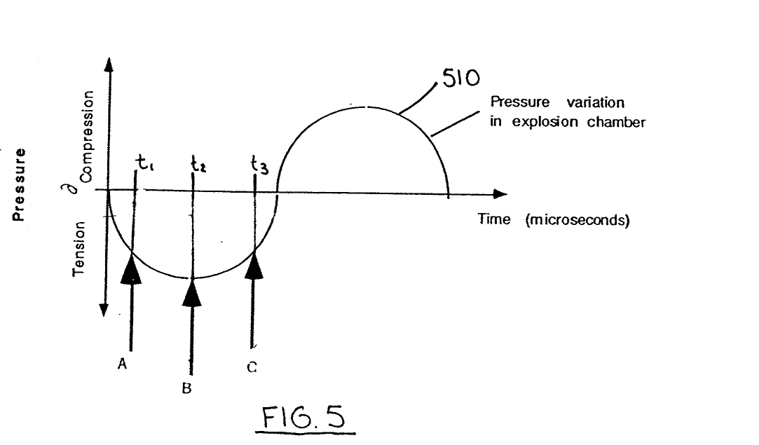

FIG. 5 illustrates how the degree of liquid pretensioning at the time of initiation of nucleation can be selected using an oscillating pressure field apparatus such as an acoustical source. The timing of the explosive burst vaporization is coordinated by synchronizing the driving pressure field and actuation of the cavitation initiation source 105, such as a neutron source, or other particle or field pulses. The sinusoidal pressure curve 510 depicts pressure as a function of time at a given volume within a chamber, such as an explosion chamber. The pressure is seen to oscillate sinusoidally between tension and compression states. For example, at time t₁, shown as “A” in FIG. 5, the pressure field is seen as descending, becoming more tensive in the next instant of time. Initiation while the pressure field is in this state will produce moderate explosive growth. Initiation at time t₂, shown as “B” will produce maximum explosive growth as the pressure field is in the maximum pretensioned state of the working liquid. Initiation at time t₃, shown as “C” will produce minimum explosive growth as the pressure field is increasing. Times corresponding to compressive values of the pressure field are not useful for pretensioning as they are compressive.

Once the cavitation initiation source 105 is activated at a pre-determined phase of the oscillating pressure field during the tensile phase, the structure for placing the working liquid in a metastable state 120 is preferably cut-off, resulting in obtaining maximum explosive vaporization. Such a timed cutoff permits flash vaporization to proceed without arrest to a level determined by the degree of pretension induced prior to cavitation. For example, at the time t₂ shown, the pressure field is cut-off at a time approximately coincident with application of the cavitation initiation signal.

In some applications, it may be desirable to produce burst implosions rather than burst explosions. Implosion can be produced using at least two methods. In the first method, an oscillating pressure field is used such that the increasing radius of the bubble is arrested and then reversed. For example, If the oscillatory pressure field is not cut-off quickly after the cavitation source energy is applied to the working liquid to generate bubbles having a bubble radius greater than the critical bubble radius, the nucleated vapor cavities will generally grow until the fluid field pressure reaches the compressive stage. The reversal and resulting collapse generally takes place very rapidly.

A second method for producing implosions utilizes constrictive bubble collapse. For example, applied to a centrifugal tensioning apparatus 300 used for projectile launch, if no constraint is applied to the working fluid as it expands after bubble nucleation, a projectile will generally be launched by the mechanical work performed by the expanding bubbles. However, if the expanding cavitation bubbles are forced to pass through a substantial constriction, the constriction can result in back pressure sufficient to cause the cavitation bubbles to collapse and cause implosion.

If implosion is generated, implosion of cavitation bubbles can produce shock pressures in the GPa range, and ultra high temperatures that can be high enough to emit visible light flashes. The micro and nano sized focused energy regions can achieve temperatures ranging from approximately 100,000 to 100,000,000 K. Such conditions are generally sufficient to destroy most living cells, such as cancerous cells.

Several operational considerations deserve mention. The energy imparted to a metastable fluid, such as a pretensioned liquid, may experience potential losses. However, if the metastable state is achieved using a centrifugal apparatus, the metastable energy state should not change as long as the rotation is proceeding. For a thermo-cycled metastable states, the extent of tension would vary depending on thermal energy exchange between the liquid its surroundings. Proper insulation or thermal management could minimize potential energy losses. For such a system, it is expected that using established heat management technology for maintaining of pretension should be possible with minimal fluctuation. For an acoustically induced metastable states as long as the acoustic energy source is active the system is ready for use.

Power supply requirements for obtaining and maintaining pretensioned metastable states will necessarily be different depending on the method used for attaining the desired pre-explosion state. An example is provided for a gun where a 9 kg projectile is to be fired at approximately 1650 m/s using the centrifugal apparatus, such as the system is shown in FIG. 3. The kinetic energy of the 9 kg projectile is approximately 12 MJ. Assuming a 30% conversion efficiency and a limit of 20 MJ/kg as energy content for pretensioned pure water, a propellant supply of approximately 2 kg (or 2 liters) will be needed.

The system can be configured such that the drive motor shaft and propulsion system are in direct contact continuously. Alternatively, the drive motor shaft may include an inertial source much like a flywheel where stored energy can be coupled to an otherwise static gun system with a clutch-type arrangement. The power supply could consist of a structure to spin a motor shaft and assembly such that rotational energy would be imparted after charging the system with propellant and appropriate placement of the projectile. The energy supply for such a system is expected to be about 40 MJ, assuming minimal losses from electrical to mechanical energy conversion in the motor-drive. This energy amount is in the range of the amount of chemical energy available from 1 kg (about 1 L) of gasoline. The energy to be imparted could be provided over a period of time if the entire gun chamber is to be brought up to speed, or delivered by a flywheel in which mechanical energy was previously stored for relatively immediate release on demand.

The potential applications for the invention are numerous. Below is a list, without limitation, of several possible applications for the invention.

The invention can be used to produce improved firearms having pressure profile control & enhanced timing control. The invention can be used for gun and artillery systems of various calibers or explosives which can be variably controlled to provide significantly higher energetic bursts than available from conventional systems. For a typical gun, it is desirable to provide energy to the bullet over a ballistic cycle being approximately 10 ms. As discussed earlier, the generation of explosive bursts depends on a combination of factors, including the metastable state and the cavitation initiation source parameters used, and the working liquid (propellant). Appropriate choice of system parameters can readily meet the 10 ms nominal requirement.

The invention suitable for use in gun and artillery systems because of the ability to control the explosive energy thrust released through appropriate system parameter selection. As a result of being able to control projectile range through controllable thrust levels, angular adjustments of apparatus such as cannons which are required in conventional systems to produce different projectile ranges will not be required with the invention.

The invention permits bodily fluids to cavitate at significantly reduced tensile pressures and as such, provides an improved method for treating tumors, cancer cells, kidney stones, and in general chem-biological agents with modest shock pressures in the 0.7 MPa (100 psi) range or less. This value may be compared to common lithotripters which cavitate bodily fluids by imposing very large shock pressures in the range of 70 Mpa (10,000 and higher psi range). This represents approximately a 100 fold decrease, or more, from shock pressures produced by conventional lithotriptcy equipment. As a result, damage to surrounding tissue can be vastly reduced compared to conventional lithotriptcy.

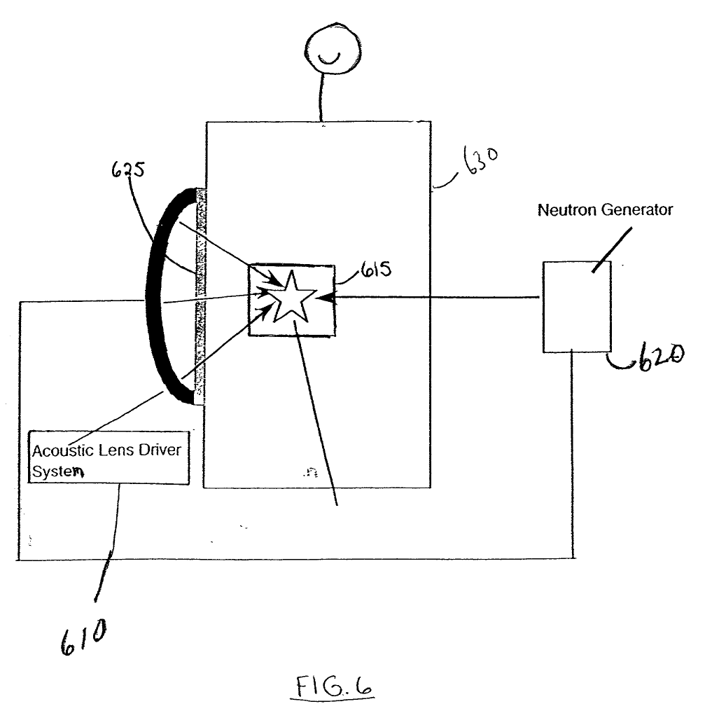

The 0.7 MPa (100 psi) or less pressures referred to above using the invention are externally-imposed acoustic pressures that are used for nucleating cavities. The GPa level pressures referred to below are pressures realized upon localized collapse of individual vapor cavities. Thus, this embodiment of the invention combines modest 0.7 MPa (100 psi) or less externally imposed pressures with cavitation initiation sources, such as nuclear (e.g. neutron), mechanical source or laser. FIG. 6 shows a schematic representation of a controllable acoustic lens neutron system for medical applications.

System 600 includes an acoustic lens driver 610 to produce a tension state in a focused region of a fluid volume 615 within body 630. Region 615 can include biological specimen, such as cancer cells or various other growths or chemical agents. Acoustic lens driver system 610 together with a focusing means, such as a parabolic-type reflector (not shown), imparts a tension state at region 615, the tension state being below a cavitation threshold of the bodily liquid. Impedance matching structure 625 functions to minimize losses from reflections and overlap of incident energy when acoustic pressure energy is transferred from driver system 610 to the ultimate medium (in the body) through a separate connecting material. The impedances, being density multiplied by the velocity of sound, of the connecting fluid and the bodily fluids is preferably comparable or otherwise close to each other to limit reflective energy losses.

Neutron generator 620 provides energy to region 615 sufficient to bubble nucleate at least one bubble having a bubble radius greater than a critical bubble radius of the bodily fluid in region 615, the formation of the bubble releasing at least a portion of said energy stored in said tension state. As described earlier, the acoustic driver source 610 is generally not shut off as neutrons are applied to region 615 by neutron generator 620. To produce implosive bubbles collapse and resulting shock pressures in the Gpa range and ultra high temperatures of up to approximately 100,000,000 K in small volumes, such as 1 μm to 10 μm, bubbles initially formed by neutron radiation while the pressure field is in the tensile phase implode when the pressure field reaches the compressive phase.

The invention can also be used to neutralize chemical and biological agents in bulk quantities, rather than through treatment of discrete surfaces in conventional systems. A system comparable to 600 can easily be adapted for this purpose.

The invention can also be used to form pulse generators and switching systems. Molecular level, nano-timed mechanical pulse generator can be formed. Such systems could be used as switching systems in confined spaces, such as for controlling fluid transport on molecular level electronic components and also for MEMS devices. Coupled with suitable transducers to transform pressure waves to electromagnetic or optical signals, the invention can be used to form electromagnetic or optical pulses which can be used in a variety of applications.

The invention can be used to provide molecular level switches. For example, for MEMS or smaller devices where fluid transport via micro or lower-type dimension orifices is needed, the invention can be used to pretension the fluid near the opening using an acoustic lens and then subjecting the region to a nucleating agent such as a fast neutron or a dissolved emitter. Upon nucleation and growth, a mechanical hammer type force can result in the vicinity of the sub-micron or other dimension type penetration. The pressure wave generated upon growth and collapse of bubbles could then be used to overcome the surface tension forces preventing the flow of liquids across the small orifice.

### EXAMPLES

20 Proof-of-principle experiments with organic and inorganic liquids have been conducted for placing various liquids under tension and then perturbing the system using fundamental particles including neutrons from small hand-portable neutron sources and other methods to initiate explosive vaporization within nanoseconds. Various metastable states have been created and demonstrate controlled explosive burst generation using a range of neutron energies as well as with dissolved emitters.

It has been demonstrated that controlled explosive bursts can be generated and coupled to launch projectiles, and also to cause shock-burst type effects. The experimental work was performed in both a static environment, such as a fluid field where the pressure state is not changing and in a dynamic environment where the pressure state is changing continuously at high frequencies (e.g., 10-20 kHz).

Experiments were conducted with a spinner arrangement using a working liquids, such as ethanol or acetone. Upon reaching a certain state of pretension the system was nucleated with an external fast neutron source (Pu—Be). The vaporization of fluid in the central bulb caused a fast pressure surge that ejected a projectile.

In another example, 20 kHz experiments were performed using a glass chamber filled with acetone. The cavitation initiating source for nucleated vapor cavities used was a neutron source, such as a Pu—Be neutron source or a pulsed neutron generator. The vapor cavity growth produced measured power surges of close to 5-10 kW even though the driving power from the 20 kHz drive transducers was only in the 1-5W range. Similar experiments were performed using C₂Cl₄ as the working liquid with similar results.

While the preferred embodiments of the invention have been illustrated and described, it will be clear that the invention is not so limited. Numerous modifications, changes, variations, substitutions and equivalents will occur to those skilled in the art without departing from the spirit and scope of the present invention as described in the claims.

### REFERENCES CITED

1. R. P. Taleyarkhan and F. Moraga, “Static and Transient Cavitation Threshold Measurements for Mercury with Dissolved Air and Helium,” Nucl. Eng. Design Journal, Vol. 207, 2001.

2. Moraga, F., R. P. Taleyarkhan, R. T. Lahey, Jr., and F. Bonetto, “Role of Very High Frequency Excitation in Single Bubble Sonolumiscence,” Journal of American Physical Society, Physics Review Letters, 2000.

3. F. Moraga, R. P. Taleyarkhan, R. T. Lahey, Jr., and F. Bonetto, “Experimental and Analytical Investigations on Role of Multi-Frequency Excitation on Enhancing Sonoluminescence in Air-Water Mixtures,” Proc. Intl. Conference on Multiphase Systems, ICMS'2000, June, 2000.

4. R. P. Taleyarkhan, F. Moraga, C. D. West, “Experimental determination of cavitation thresholds in liquid water and mercury,” Proceedings of the 2nd international topical meeting on accelerator applications (AccApp'98), Gatlinburg, Tenn., USA (September, 1998).

5. Roi, N., “The Initiation and Development of Ultrasonic Cavitation,” Soviet Physics, Vol.3, No.1, 1957.

6. Speedy, R. J., “Journal of Physical Chemistry,”, Vol. 86, No.982, 1982.

7. C. D. West and R. Howlett, “Experimental measurements on cavitation bubble dynamics,” Acustica, Vol.21, (1969).

8. For Acetone: B. Hahn, “The fracture of liquids under stress due to ionizing particles,” Nuovo Cimento, Vol.22, (1961).

9. Moraga, F., R. P. Taleyarkhan, R. T. Lahey, Jr., and F. Bonetto, “Role of Very High Frequency Excitation in Single Bubble Sonolumiscence,” Journal of American Physical Society, Physics Review Letters, 2000.

We claim:

1. A burst generator, comprising:
structure for placing at least a portion of a liquid into a tension state, said tension state being below a cavitation threshold of said liquid, said tension state imparting stored mechanical energy into said liquid portion;
structure for cavitating said liquid portion sufficient to bubble nucleate at least one bubble having a bubble radius greater than a critical bubble radius of said liquid, formation of said bubble releasing at least a portion of said energy stored in said tension state.

2. The burst generator of claim 1, wherein said structure for placing a liquid under tension is a centrifugal source.

3. The burst generator of claim 1, wherein said structure for placing a liquid under tension is an acoustic wave source.

4. The burst generator of claim 3, wherein said acoustical wave source includes an acoustical wave focusing device.

5. The burst generator of claim 4, wherein said focusing device is a parabolic-type reflector.

6. The burst generator of claim 1, wherein said structure for placing said liquid under tension is a magnetostrictive source.

7. The burst generator of claim 1, wherein said structure for placing said liquid under tension is an electrostrictive (piezoelectric) source.

8. The burst generator of claim 1, wherein said structure for cavitating is an acoustical source.

9. The burst generator of claim 1, wherein said structure for placing said liquid under tension is an acoustical source, said acoustical source also being said structure for cavitating.

10. The burst generator of claim 1, wherein said structure for cavitating is a source of fundamental particles, said source of fundamental particles being at least one selected from the group consisting of alpha emitters, neutron sources and fission fragment sources.

11. The burst generator of claim 1, wherein said structure for cavitating comprises at least one selected from a laser source and a mechanical source.

12. The burst generator of claim 1, wherein said structure for cavitating is neutron source, said neutron source being an isotopic source having at least one shutter, said shutter opened to synchronize neutron impact with a location in said liquid when said liquid is at a predetermined liquid tension level.

13. The burst generator of claim 1, wherein said structure for cavitating is an alpha particle source.

14. The burst generator of claim 1, wherein said liquid is at least one selected from the group consisting of water, mercury, acetone, tetrachloroethylene, acetophenone and glycols.

15. The burst generator of claim 1, wherein said liquid is a biological liquid, said biological liquid selected from the group consisting of blood, synovial liquid, mucus and urine.

16. The burst generator of claim 1, wherein said energy stored in said tension state is released no more than about 1.0 μsec following receipt of cavitation initiation energy from said structure for cavitating.

17. The burst generator of claim 1, wherein said structure for placing a liquid into a tension state includes structure for controlling a tension level of said tension state.

18. The burst generator of claim 1, further comprising a structure for generating an oscillatory pressure field in said liquid, a compressive phase of said pressure field for imploding at least one of said bubbles.

19. The burst generator of claim 1, further comprising a structure for condensing said vapor back into a liquid state following formation of said bubbles.

20. The burst generator of claim 1, wherein said burst generator further includes a controller for synchronizing delivery of at least one cavitation signal from said structure for cavitating at a predetermined location in said liquid having a predetermined tension level.

21. The burst generator of claim 20, wherein said structure for placing a liquid into a tension state produces a time varying tension level in said liquid.

22. The burst generator of claim 1, further comprising a cooling device for reducing a temperature of said liquid below ambient temperature.

23. A burst generator, comprising:
a container for confining a liquid;
structure for placing at least a portion of said liquid into a deep metastable state, and
structure for cavitating at least a portion of said liquid sufficient to bubble nucleate at least one bubble having a bubble radius greater than a critical bubble radius of said liquid.

24. The burst generator of claim 23, wherein said structure for placing at least a portion of said liquid into a deep metastable state is an acoustical source and said structure for cavitating is a neutron source.

25. An armament, comprising:
a substantially enclosed container having at least one projectile and a liquid;
structure for placing said liquid into a tension state, said tension state being below the cavitation threshold of said liquid, said tension state imparting stored mechanical energy into said liquid, and
structure for cavitating at least a portion of said tensioned sufficient to bubble nucleate at least one bubble having a bubble radius greater than a critical bubble radius of said liquid, formation of said bubble releasing at least a portion of said energy stored in said tension state, whereby an explosive burst results which propels said projectile out of said container.

26. The armament of claim 25, wherein said projectile includes an explosive.

27. The armament of claim 25, wherein said armament is a gun or a rifle.

28. The armament of claim 25, further comprising a structure for controlling thrust, wherein said armament develops a controllable thrust within said container, whereby different remote distances can be reached by said projectile without adjusting a firing angle of said armament.

29. The armament of claim 28, wherein said structure for controlling thrust controls a level of said tension state.

30. The armament of claim 28, wherein said structure for controlling thrust controls energy released by said structure for cavitating.

31. The armament of claim 25, wherein said structure for cavitating is a source of fundamental particles.

32. The armament of claim 25, wherein said structure for cavitating is a neutron source.

33. The armament of claim 25, wherein said liquid is at least one selected from the group consisting of water, mercury, acetone, tetrachloroethylene, acetophenone and glycols.

34. The armament of claim 25, further comprising a structure for condensing said vapor back into a liquid state following bubble formation.

35. The burst generator of claim 25, wherein said armament further includes a controller for synchronizing delivery of at least one initiation signal from said structure for cavitating with a desired tension level in said liquid.

36. The armament of claim 25, wherein said structure for placing a liquid into a tension state produces a time varying tension level in said liquid.

37. The armament of claim 25, further comprising a cooling device for reducing a temperature of said liquid below ambient temperature.

38. A medical device, comprising:
structure for placing a bodily liquid region contained within a body into a tension state, said tension state being below the cavitation threshold of said bodily liquid and imparting stored mechanical energy into said liquid in said region;
structure for initiating cavitation of at least a portion of liquid in said region into at least one bubble while said liquid in said region is in said tension state; and
structure for applying a compressive wave to at least one of said bubbles.

39. The medical device of claim 38, wherein structure for placing a liquid region contained within a body into a tension state and said structure for applying a compressive wave to at least one of said bubbles are supplied by a single source of an oscillatory pressure field.

40. The medical device of claim 38, wherein said structure for placing a liquid into an oscillatory pressure state is an acoustic wave source.

41. The medical device of claim 40, wherein said acoustical wave source includes an acoustical wave focusing device.

42. The medical device of claim 41, wherein said device further comprises a parabolic-type reflector.

43. The medical device of claim 38, wherein said structure for structure for initiating cavitation comprises an acoustical source.

44. The medical device of claim 38, wherein said structure for initiating cavitation is a source of fundamental particles, said source of fundamental particles being at least one selected from the group consisting of alpha emitters, neutron sources and fission fragment sources.

45. The medical device of claim 38, wherein said neutron source is an isotopic source with at least one shutter, wherein said neutron source is an isotopic source having at least one shutter, said shutter opened to synchronize neutron impact with a location in said bodily liquid having a predetermined liquid tension level.

46. The medical device of claim 45, wherein said predetermined liquid tension level is approximately a maximum tension level provided by said liquid.

47. The medical device of claim 38, wherein said bodily liquid is at least one selected from the group consisting of blood, synovial liquid, mucus and urine.

48. The medical device of claim 38, wherein said structure for placing a bodily liquid into a tension state includes structure for controlling a level of said tension state.

49. The medical device of claim 38, wherein said structure for placing a bodily liquid region contained within a body into an oscillating pressure state comprises a plurality of oscillatory pressure sources.

50. The medical device of claim 38, wherein said structure for initiating cavitation comprises at least one selected from a laser source and a mechanical source.

51. A pulse generator, comprising:
a container for containing a liquid;
structure for placing a liquid into a tension state, said tension state being below the cavitation threshold of said liquid, said tension state imparting stored mechanical energy into said liquid;
structure for cavitating at least a portion of said tensioned liquid sufficient to bubble nucleate at least one bubble having a bubble radius greater than a critical bubble radius of said liquid, formation of said bubble releasing at least a portion of said energy stored in said tension state.

52. The pulse generator of claim 51, wherein said structure for placing a liquid under tension is an acoustic wave source.

53. The pulse generator of claim 51, wherein said structure for placing said liquid under tension is an acoustical source, said acoustical source also being said structure for cavitating.

54. The pulse generator of claim 51, wherein said structure for cavitating is a source of fundamental particles, said source of fundamental particles being at least one selected from the group consisting of alpha emitters, neutron sources and fission fragment sources.

55. The pulse generator of claim 51, wherein said structure for cavitating comprises at least one selected from a laser source and a mechanical source.

56. The pulse generator of claim 51, wherein said structure for cavitating is a neutron source, said neutron source being an isotopic source with at least one shutter, said shutter adapted to open to synchronize neutron impact with a location in said liquid having a predetermined liquid tension level.

57. The pulse generator of claim 51, wherein said liquid is at least one selected from the group consisting of water, mercury, acetone, tetrachloroethylene, acetophenone and glycols.

58. The pulse generator of claim 51, wherein said structure for placing a liquid into a tension state includes structure for controlling a level of said tension state.

59. The pulse generator of claim 51, further comprising a structure for converting said bursts into electromagnetic signals.

60. The pulse generator of claim 51, wherein said bursts are directed to propel a liquid through an orifice, said orifice being no larger than micron scale.

61. A method for producing energetic bursts, comprising the steps of:
placing a liquid into a tension state, said tension state being below the cavitation threshold of said liquid, said tension state imparting stored mechanical energy into said liquid;
cavitating at least a portion of said tensioned liquid sufficient to bubble nucleate at least one bubble having a bubble radius greater than a critical bubble radius of said liquid, formation of said bubble releasing at least a portion of said energy stored in said tension state.

62. The method of claim 61, wherein a centrifugal source is used for said tensioning.

63. The method of claim 61, wherein an acoustic wave source is used for said tensioning.

64. The method of claim 63, further comprising the step of focusing said acoustical waves.

65. The method of claim 64, wherein a parabolic-type reflector is used for said focusing.

66. The method of claim 61, wherein a source of fundamental particles is used for said cavitating step.

67. The method of claim 61, wherein a neutron source is used for said cavitating step, said neutron source for emitting high energy neutrons.

68. The method of claim 67, further comprising the step of synchronizing neutron impact with a location having a predetermined liquid tension level.

69. The method of claim 61, wherein said liquid is at least one selected from the group consisting of water, mercury, acetone, tetrachloroethylene, acetophenone and glycols.

70. The method of claim 61, further comprising the step of imploding said bubbles.

71. The method of claim 70, wherein an externally applied pressure field is used to generate a compressive pressure field in said liquid for said imploding step.

72. The method of claim 61, further comprising the step of condensing said vapor back into said liquid.

73. The method of claim 61, further comprising the step of synchronizing delivery of at least one initiation signal with a desired tension level in said liquid.

74. The method of claim 61, further comprising the step of cooling said liquid below ambient temperature.

75. The method of claim 61, wherein a laser source or a mechanical source is used for said cavitating step.
